(19) United States
(12) Patent Application Publication
(10) Pub. No.: US 2010/0287884 A1
(43) Pub. Date: Nov. 18, 2010

(54) POWDER CONDITIONING OF UNIT DOSE DRUG PACKAGES

(76) Inventors: Sangita Seshadri, Saratoga, CA (US); Barry Fong, San Lorenzo, CA (US); Srinivas Palakodaty, Foster City, CA (US); Concordio Candug Anacleto, Daly City, CA (US); Patrick Reich, San Jose, CA (US); Andrew John Boeckl, Amsterdam (NL); Derrick J. Parks, Belmont, CA (US); Gordon Stout, El Cerrito, CA (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/738,607

(22) PCT Filed: Oct. 23, 2008
(86) PCT No.: PCT/US08/12117

§ 371 Date: Jul. 7, 2010

Related U.S. Application Data
(60) Provisional application No. 61/000,627, filed on Oct. 25, 2007.

(51) Int. Cl. A61M 15/00; B65B 1/22

(52) U.S. Cl. 53/437; 53/525; 128/203.12

(57) ABSTRACT

The invention provides techniques for treating or conditioning powders subsequent to their packaging to facilitate extraction of the powders from their packaging.

### CROSS REFERENCE TO RELATED APPLICATIONS

This application claims the benefit of priority under 35 U.S.C. §119(e) to Provisional Application Ser. No. 61/000,627, filed 25 Oct. 2007, which is incorporated herein by reference in its entirety.

### FIELD OF THE INVENTION

This invention provides (among other things) means for conditioning powder compositions in blisters or other configurations to improve dispersibility of the powder. The invention also provides various apparatuses to achieve the same.

### BACKGROUND OF THE INVENTION

The need for effective therapeutic treatment of patients has resulted in the development of a variety of techniques for delivering a pharmaceutical formulation to a patient. One traditional technique involves the oral delivery of a pharmaceutical formulation in the form of a pill, capsule, or the like. Inhaleable drug delivery, where an aerosolized pharmaceutical formulation is orally or nasally inhaled by a patient to deliver the formulation to the patient's respiratory tract, has also proven to be an effective manner of delivery. In one inhalation technique, a pharmaceutical formulation is delivered deep within a patient's lungs where it may be absorbed into the blood stream. In another inhalation technique, a pharmaceutical formulation is delivered to a targeted region in the respiratory tract to provide local treatment to the region. Many types of inhalation devices exist including devices that aerosolize a dry powder pharmaceutical formulation.

The pharmaceutical formulation is often packaged so that it may be made easily available to a user. For example, a dose or a portion of a dose may be stored between layers of a multi-layered package, conventionally referred to as a blister or blister pack. Typically, a cavity is formed in a lower layer, the pharmaceutical formulation is deposited within the cavity, and an upper layer is sealed onto the lower layer, such as by heating and/or compressing the layers, to secure the pharmaceutical formulation within the cavity. Alternatively, the dose may be stored in a capsule that is to be swallowed or from which the pharmaceutical formulation may be aerosolized. Other packages, such as bottles, vials, and the like, may also be used to store the pharmaceutical formulation. PCT application WO01/43802 discloses systems and methods for treating packaged powders at the time of inhalation.

It is often difficult to effectively fill packages with the pharmaceutical formulation. For example, during some powder filling process, it is difficult to sufficiently fluidize the powder and/or to maintain consistent flow properties of the powder. On the other hand, sometimes the powder may be compacted into ‘pucks’ for filling into formed blisters. Depending on the bulk powder characteristics, the vacuum and the ultrasonic probe amplitude on the filler are adjusted to form the puck to give the desired control over the fill mass. The puck may break down into powder during subsequent operations on the filler/packager or during transport. However, on occasions when the puck is relatively ‘hard’, it may not completely disperse into a uniform powder for its intended delivery. Mechanical vibrations during subsequent shipping of the final product could have effect on the powder in the blister pack. This may result in variable doses to the patient as the emitted dose results vary from the end of manufacturing release test to the time of dosing. It is, therefore, useful to ‘condition’ or break the powder puck after filling and sealing the blister to ensure a consistent product performance from the time of manufacture to the time of dosing. Therefore, there is a need in the filed, to develop novel mechanisms to condition the powders.

### SUMMARY OF THE INVENTION

The invention provides techniques for treating or conditioning powders subsequent to their packaging to facilitate extraction of the powders from their packaging. These and other objects, aspects, embodiments and features of the invention will become more fully apparent when read in conjunction with the following detailed description.

### BRIEF DESCRIPTION OF THE FIGURES

FIG. 1 shows a web whacker.

FIG. 2 shows an acoustic speaker on a filler/packager.

FIGS. 3A and 3B show ultrasonic conditioning of blisters.

FIG. 4 shows an ultrasonic bath with blisters.

FIG. 5 shows the effect of various conditioning methods on emitted dose and blister retention.

FIG. 6 shows the effect of ultrasonic energy on conditioning.

FIG. 7 shows the effect of ultrasonic conditioning on shipped and unshipped blisters at various energy levels.

FIG. 8 shows the effect of ultrasonic conditioning on bulk shipped and unshipped blisters.

### DETAILED DESCRIPTION OF THE INVENTION

It must be noted that, as used in this specification, the singular forms “a,” “an,” and “the” include plural referents unless the context clearly dictates otherwise.

In describing and claiming the present invention, the following terminology will be used in accordance with the definitions described below.

### Definitions

The terms used in this disclosure are defined as follows unless otherwise indicated. Standard terms are to be given their ordinary and customary meaning as understood by those of ordinary skill in the art, unless expressly defined herein.

Term “conditioning” is used to describe processes to facilitate a more uniformly dispersible powder that exhibits less agglomeration compared to powders that are not conditioned. “Deagglomeration” is used interchangeably to mean conditioning.

A composition that is “suitable for pulmonary delivery” refers to a composition that is capable of being aerosolized and inhaled by a subject so that a portion of the aerosolized particles reaches the lungs, e.g., to permit entry into the alveoli and into the blood. Such a composition may be considered “respirable” or “inhaleable.”

An “aerosolized” composition contains solid particles that are suspended in a gas (typically air), typically as a result of actuation (or firing) of an inhalation device. A passive dry powder inhaler would be actuated by a user's breath.

A “dry powder inhaler” is a device that is loaded with a unit dose reservoir (e.g., a blister), of the drug in powder form. Depending on the treatment regimen, more than one unit dose may need to be delivered to a subject in need thereof. Generally, the inhaler is activated by taking a breath. For example, a capsule or blister is punctured and the powder is dispersed so that it can be inhaled, e.g., in a “Spinhaler” or “Rotahaler.” “Turbohalers” are fitted with canisters that deliver measured doses of the drug in powder form.

As used herein, the term “emitted dose” or “ED” refers to an indication of the delivery of dry powder from an inhaler device after an actuation or dispersion event from a powder unit or reservoir. ED is defined as the ratio of the dose delivered by an inhaler device to the nominal dose (i.e., the mass of powder per unit dose placed into a suitable inhaler device prior to firing). The ED is an experimentally determined amount, and may be determined using an in vitro device set up which mimics patient dosing. To determine an ED value, as used herein, dry powder is placed into a device to be tested. The device is actuated (e.g., by inserting a blister, rotating a mouthpiece of the device, and applying a 30 L/min vacuum source to an exit of the mouthpiece), dispersing the powder. The resulting aerosol cloud is then drawn from the device by vacuum (30 L/min) for 2.5 seconds after actuation, where it is captured on a tared glass fiber filter (Gelman, 47 mm diameter) attached to the device mouthpiece. The amount of powder that reaches the filter constitutes the delivered dose. For example, for a capsule containing 5 mg of dry powder that is placed into an inhalation device, if dispersion of the powder results in the recovery of 4 mg of powder on a tared filter as described above, then the ED for the dry powder composition is 80% (=4 mg (delivered dose)/5 mg (nominal dose)).

A composition in “dry powder form” is a powder composition that typically contains less than about 20% moisture, or less than about 10% moisture, or less than about 5% moisture, or less than about 3% moisture, or less than about 1% moisture.

As used herein, “mass median diameter” or “MMD” refers to the median diameter of a plurality of particles, typically in a polydisperse particle population, i.e., consisting of a range of particle sizes. MMD values as reported herein are determined by laser diffraction (Sympatec Helos, Clausthal-Zellerfeld, Germany), unless the context indicates otherwise. Typically, powder samples are added directly to the feeder funnel of the Sympatec RODOS dry powder dispersion unit. This can be achieved manually or by agitating mechanically from the end of a VIBRI vibratory feeder element. Samples are dispersed to primary particles via application of pressurized air (2 to 3 bar), with vacuum depression (suction) maximized for a given dispersion pressure. Dispersed particles are probed with a 632.8 nm laser beam that intersects the dispersed particles' trajectory at right angles. Laser light scattered from the ensemble of particles is imaged onto a concentric array of photomultiplier detector elements using a reverse-Fourier lens assembly. Scattered light is acquired in time-slices of 5 ms. Particle size distributions are back-calculated from the scattered light spatial/intensity distribution using an algorithm.

“Mass median aerodynamic diameter,” or “MMAD,” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density, and physical size of a particle. As used herein, MMAD refers to the midpoint or median of the aerodynamic particle size distribution of an aerosolized powder determined by cascade impaction at standard conditions (20° C.; 40% RH) using the device to be tested.

“Fine particle fraction” is the fraction of particles with an aerodynamic diameter that is less than 5 microns (μm). Where specified, the fine particle fraction may also refer to the fraction of particles with an aerodynamic diameter that is less than 3.3 microns. “Receptacle” is a container. For example, a receptacle may be a unit dose receptacle, or it may be a reservoir having multiple doses. Examples of unit dose receptacles include blister packs and capsules. In certain embodiments, the receptacle may be removable from an inhaler device, or the receptacle may be part of an inhaler device. The receptacle typically comprises any material that allows tearing, e.g., a controlled tearing, such as foil-plastic laminates or other materials. Examples of containers/receptacles include, but are not limited to, capsules, blisters, vials, or container closure systems made of metal, polymer (e.g., plastic, elastomer), glass, or the like.

“Receptacle” is a container. For example, a receptacle may be a unit dose receptacle, or it may be a reservoir having multiple doses. Examples of unit dose receptacles include blister packs and capsules. In certain embodiments, the receptacle may be removable from an inhaler device, or the receptacle may be part of an inhaler device. The receptacle typically comprises any material that allows tearing, e.g., a controlled tear, such as foil-plastic laminates.

In one embodiment, the invention comprises a web whacker or a mechanical striker that comprises of collapsible, rotatable arms on a somewhat circular shaft. The rotatable arm is connected to a motor. The rotatable arm may comprise of plurality of protrusions. The arm strikes the web (a blister comprising one or more individual unit drug dose in a receptacle). The strike may be to the side of the web or from the top or the bottom of the web depending on the configuration of the arm. The rotation speed of the shaft and the duration between each ‘draw’ on the packaging line determines the degree of puck break up. The rotatable arm may rotate at frequencies from about 500 rotations per minute (rpm) to about 4000 rpm. The duration of subjecting the web to whacking is a balance between production capacity (draw time) and efficiently breaking the puck into dispersible powder.

In a second embodiment, called acoustic conditioning, the web containing the sealed blisters is subjected to mechanical vibration by an acoustic speaker before it is drawn and punched into individual blisters. The speaker may be located above, blow or to side of the web. More than one speaker may be placed in different configurations to optimize the conditioning process (e.g. two speakers facing the web on either side). The vibration of the web can be adjusted by tuning the frequency and amplitude of the speaker which in turn is controlled by the voltage applied to the speaker coil. The duration of subjecting the web to acoustic vibration is a balance between production capacity (draw time) and efficiently breaking the puck into dispersible powder.

In a third embodiment, called ultrasonic conditioning, the web containing the sealed blisters is subjected to mechanical vibration by an ultrasonic probe (or an ultrasonic horn) before it is drawn and punched into individual blisters. The probe may be located beneath, top or on the side of the web. The vibration of the web can be adjusted by tuning the amplitude of the ultrasonic probe at a fixed frequency. The vibration frequency may range from about 5 kHz to about 100 kHz, preferably from about 10 kHz to about 40 kHz. The efficiency of breaking the puck depends on coupling the probe with the web. The vibration amplitude may range from about 0.001 inch to about 0.01 inch. The ultrasonic probe may be used for a variable period of time. It may be used from about 0.1 second to about 3 seconds, preferably from about 0.25 second to about 2 seconds. The ultrasonic probe may The duration of subjecting the web to ultrasonic probe is a balance between production capacity (draw time) and efficiently breaking the puck into dispersible powder. The flexibility of this approach is that the probe could be located either beneath, top or on the side of the web.

In a further embodiment, the web or the blisters may be positioned using a cross beam horizontally arranged transverse to the web running direction, which is vertically positionable; and a plurality of plugs structured and arranged on the crossbeam, wherein the cross beam has an engaged position structured and arranged so that the plurality of plugs may contact at least one of the web and the probe tips, and a disengaged position structured and arranged so that the plurality of plugs may not contact the web or the probe tips.

In another embodiment, the web or the blisters may be positioned by using spring fingers on a rotatable shaft running transverse to the web running direction. The spring finger may further comprise of plastic or rubber tip to reduce the noise and aid in smooth operation. The spring fingers may be appended to a roller with bearings to facilitate operation. The ultrasonic treatment of some powders may lead to transient tribo-charging. A short period of storage before using the blisters may be required for relaxation.

In a fourth embodiment, also called ultrasonic conditioning, the web containing the sealed blisters is subjected to mechanical vibration by an ultrasonic bath before it is drawn and punched into individual blisters.

The powder may be initially stored in the sealed receptacle, which is opened prior to aerosolization of the powder, as described in U.S. Pat. No. 5,785,049, U.S. Pat. No. 5,415,162 and U.S. patent application Ser. No. 09/583,312. Alternatively the powder may be contained in a capsule, as described in U.S. Pat. No. 4,995,385, U.S. Pat. No. 3,991,761, U.S. Pat. No. 6,230,707, and PCT Publication WO 97/27892, the capsule being openable before, during, or after insertion of the capsule into an aerosolization device. In the bulk, blister, capsule, or the like form, the powder may be aerosolized by an active element, such as compressed air, as described in U.S. Pat. No. 5,458,135, U.S. Pat. No. 5,785,049 and U.S. Pat. No. 6,257,233, or propellant, as described in U.S. patent application Ser. No. 09/556,262, filed on Apr. 24, 2000, and entitled “Aerosolization Apparatus and Methods”, and in PCT Publication WO 00/72904. Alternatively the powder may be aerosolized in response to a user's inhalation, as described for example in the aforementioned U.S. patent application Ser. No. 09/583,312 and U.S. Pat. No. 4,995,385. All of the above references being incorporated herein by reference in their entireties.

The receptacle may be inserted into an aerosolization device. The receptacle may be of a suitable shape, size, and material to contain the pharmaceutical composition and to provide the pharmaceutical composition in a usable condition. For example, the capsule or blister may comprise a wall, which comprises a material that does not adversely react with the pharmaceutical composition. In addition, the wall may comprise a material that allows the capsule to be opened to allow the pharmaceutical composition to be aerosolized. In one version, the wall comprises one or more of gelatin, hydroxypropyl methylcellulose (HPMC), polyethyleneglycol-compounded HPMC, hydroxyproplycellulose, agar, aluminum foil, or the like. In one version, the capsule may comprise telescopically adjoining sections, as described for example in U.S. Pat. No. 4,247,066, which is incorporated herein by reference. The size of the capsule may be selected to adequately contain the dose of the pharmaceutical composition. The sizes generally range from size 5 to size 000 with the outer diameters ranging from about 4.91 mm to 9.97 mm, the heights ranging from about 11.10 mm to about 26.14 mm, and the volumes ranging from about 0.13 mL to about 1.37 mL, respectively. Suitable capsules are available commercially from, for example, Shionogi Qualicaps Co. in Nara, Japan and Capsugel in Greenwood, S.C. After filling, a top portion may be placed over the bottom portion to form a capsule shape and to contain the powder within the capsule, as described in U.S. Pat. Nos. 4,846,876 and 6,357,490, and in WO 00/07572, which are incorporated herein by reference. After the top portion is placed over the bottom portion, the capsule can optionally be banded.

Prior to use, dry powders are generally stored under ambient conditions, and preferably are stored at temperatures at or below about 25° C., and relative humidities (RH) ranging from about 30 to 60%. More preferred relative humidity conditions, e.g., less than about 30%, may be achieved by the incorporation of a desiccating agent in the secondary packaging of the dosage form.

### Devices:

The compositions of one or more embodiments of the present invention may be administered by various methods and techniques known and available to those skilled in the art.

For example, in one or more embodiments, the compositions described herein may be delivered using any suitable dry powder inhaler (DPI), i.e., an inhaler device that utilizes the patient's inhaled breath as a vehicle to transport the dry powder drug to the lungs. Preferred are Nektar Therapeutics' dry powder inhalation devices as described in U.S. Pat. Nos. 5,458,135; 5,740,794; and 5,785,049, which are incorporated herein by reference.

When administered using a device of this type, the powder is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in WO 97/41031 (1997), which is incorporated herein by reference.

Also suitable for delivering the powders described herein are dry powder inhalers of the type described, for example, in U.S. Pat. Nos. 3,906,950 and 4,013,075, which are incorporated herein by reference, wherein a premeasured dose of dry powder for delivery to a subject is contained within a hard gelatin capsule.

Other dry powder dispersion devices for pulmonarily administering dry powders include those described, for example, in EP 129985; EP 472598; EP 467172; and U.S. Pat. No. 5,522,385, which are incorporated herein by reference. Also suitable for delivering the dry powders of the invention are inhalation devices such as the Astra-Draco “TURBOHALER”. This type of device is described in detail in U.S. Pat. Nos. 4,668,281; 4,667,668; and 4,805,811, all of which are incorporated herein by reference. Other suitable devices include dry powder inhalers such as the ROTAHALER™ (Glaxo), Discus™ (Glaxo), Spiros™ inhaler (Dura Pharmaceuticals), and the Spinhaler™ (Fisons). Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572, which is incorporated herein by reference. Another class of dry powder inhalers, which may be used, is disclosed in U.S. Provisional Application Nos. 60/854,601 and 60/906,977, which are incorporated herein by reference, and which are owned by Nektar Therapeutics.

Dry powders may also be delivered using a pressurized, metered dose inhaler (MDI), e.g., the Ventolin™ metered dose inhaler, containing a solution or suspension of drug in a pharmaceutically inert liquid propellant, e.g., a chlorofluorocarbon or fluorocarbon, as described in U.S. Pat. Nos. 5,320,094 and 5,672,581, which are both incorporated herein by reference.

The pharmaceutical formulation may comprise an active agent. The active agent described herein includes an agent, drug, compound, composition of matter, or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, pulmonary system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-2 fusion protein, interleukin-1 receptor antagonist, interleukin-3, interleukin-4, interleukin-6, interleukin-11, luteinizing hormone releasing hormone (LHRH), insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulin-like growth factor binding protein (e.g., IGFBP3), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (DNase), bactericidal/permeability increasing protein (BPI), anti-CMV antibody, 13-cis retinoic acid, 9-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetonide, budesonide acetonide, fluticasone, ipratropium bromide, flunisolide, cromolyn sodium, ergotamine tartrate; rilapladib, darapladib, remogliflozin etabonate, otelixizumab, carvedilol, fondaparnux, metformin, rosiglitazone, farglitizar, sitamaquine, tafenoquine, belimumab, pazopanib, ronacaleret, solabegron, dutasteride, mepolizumab, ofatumumab, orvepitant, casopitant, firategrast, lamotrigine, ropinirole, iboctadekin, rituximab, totrombopag, lapatinib, elesclomol, topotecan, darotropium, zafirlukast, anastrozole, candesartan cilexetil, bambuterol, terbutaline, mepivacaine, bicalutamide, prilocaine, rosuvastatin, propofol, fulvestrant, isosorbide-5-mononitrate, isosorbide dinitrate, propanolol, gefitinib, enalapril, felodipine, metoprolol, omeprazole, bupivacaine, primidone, ropivacaine, esomeprazole, atenolol, nifedipine, tamoxifen, formoterol, ramipril, quetiapine, chlorthalidone, raltitrexed, viloxazine, lisinopril, hydrochlorothiazide, goserelin, zolmitriptan, saxagliptin, dapagliflozin, motavizumab, ibuprofen, ethinyl estradiol, levonorgestrel, loratadine, amiodarone, brompheniramine, dextromethorphan, phenylephrine, phenylpropanolamine, venlafaxine, etanercept, norgestrel, minocycline, gemtuzumab ozogamicin, oprelvekin, pantoprazole, promethazine, medroxyprogesterone, epinephrine, desvenlafaxine, sirolimus, temsirolimus, ethionamide, tigecycline, tazobactam, bazedoxifene, priniberel, bifeprunox, bapineuzumab, lecozotan, vabicaserin, rotigaptide, stamulumab, methylnaltrexone, bosutinib, alteplase, tenecteplase, meloxicam, tamsulosin, tiotropium, salbutamol, fenoterol, nevirapine, tipranavir, duloxetine, pramipexole, dipyridamole, naproxen, bevacizumab, sulfamethoxazole trimethoprim, benzafibrate, ibandronate, mycophenolate mofetil, enfuvirtide, trastuzumab, saquinavir, granisetron, mefloquine, levodopa benserazide, epoetin beta, filgrastim, dornase alfa, isotretinoin, oseltamivir, erlotinib, ketorolac, torasemide, valganciclovir, diazepam, tretinoin, nelfinavir, capecitabine, orlestat, daclizumab, tocilizumab, ocrelizumab, aleglitazar, pertuzumab, nicaraven, omalizumab, risedronate, fexofenadine, zolpidem, dolasetron, leflunomide, irbesartan, clindamycin, fluorouracil, leuprolide, rasburicase, oxaliplatin, hyaluronate, telithromycin, glargine, enoxaparin, ciclopirox, clopidogrel, riluzole, poly-L-lactic acid, docetaxel, alfuzosin, glimepiride, chloroquine, mepenzolate, clomiphene, desmopressin, meperidine, prednicarbate, glyburide, ergocalciferol, methanamine, hydrocortisone, betaxolol, furosemide, indapamide, ambenonium, nilutamide, metronidazole, desipramine, hydroxychloroquine, rifapentine, milrinone, diflorasone, rifampin, tiludronate, pentazocine, pentoxyifylline, hyaluronic acid, benzalkonium, tissue-plasminogen activator, CMV immune globulin, glucocerebrocidase, trimetrexate, porfimer, sterile thiotepa, amifostine, doxorubicin, 3TC, daunorubicin, cidofovir, carmustine, mitoxantrone, HIV protease inhibitor, dopamine DA1 agonist, carbamazepine, sermorelin, peptide GP IIb/IIIa antagonist, palivizumab, thalidomide, infliximab, fomivirsen, doxycycline, sevelamer, modafinil, anti-thymocyte globulin, hepatitis B immune globulin, amprenavir, cytarbine, zanamivir, bexarotene, somatropin, zonisamide, verteporfin, colesevelam, direct thrombin inhibitor, thrombin, antihemophilic factor, methylphenidate, arsenic trioxide, choriogonadotropin alpha, hyaluronan, epivir, retrovir, ziagen, bivalirudin, intron, alemtuzumab, triptorelin, nesiritide, osteogenic protein, tenofovir disoproxil, bosentan, endothelin receptor antagonist, dexmethylphenidate, 5HT 1B/1D agonist, Y2B8, secretin, treprostinil, sodium oxybate, prasterone, adefovir dipivoxil, mitomycin, adalimumab, alefacept, agalsidase beta, laronidase, gemifloxacin, tositumomab, iodine, nucleoside reverse transcriptase inhibitor, palonosetron, gallium nitrate, efalizumab, risperidone, fosamprenavir, abarelix, tadalafil, cetuximab, cinacalcet, trospium, rifaximin, azacitidine, emtricitabine, erlotinib, natalizumab, eszopiclone, palifermin, aptaninb, clofarabine, iloprost, pramlintide, exenatide, galaplase, hydralazine, sorafenib, lenalidomide, ranolazine, naltrexone, alglucosidase alfa, decitabine, ranibizumab, efavirenz, emtracitabine, idursulfase, oravescent fentanyl, panitumumab, telbivudine, aliskiren, eculizumab, ambrisentan, armodafinil, lanreotide, sapropterin, rimantidine, and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, RNAi, aptamers, siRNA, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines, such as cytomegalovirus, rabies, HIV, S. pneumoniae, Dengue fever, Epstein-Barr, West Nile, hepatitis, malaria, tuberculosis, Vericella Zoster, influenza, herpes, diphtheria, tetanus, pertussis, acellular pertussis, human papilloma, BCG, Hib-MenCY-TT, and MenACWY-TT. The active agent may also comprise antibodies, such as monoclonal antibody or monoclonal antibody fragment, such as anti-CD3 mAb, digoxin-binding ovine antibody fragment, anti-RSV Ab, anti-TAC mAb, or anti-platelet mAb. Other useful drugs include those listed within the Physician's Desk Reference (most recent edition).

As noted above, the dry powder may include one or more pharmaceutically acceptable excipient. Examples of pharmaceutically acceptable excipients include, but are not limited to, lipids, metal ions, surfactants, amino acids, carbohydrates, buffers, salts, polymers, and the like, and combinations thereof.

Examples of lipids include, but are not limited to, phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate.

In one or more embodiments, the phospholipid comprises a saturated phospholipid, such as one or more phosphatidylcholines. Exemplary acyl chain lengths are 16:0 and 18:0 (i.e., palmitoyl and stearoyl). The phospholipid content may be determined by the active agent activity, the mode of delivery, and other factors.

Phospholipids from both natural and synthetic sources may be used in varying amounts. When phospholipids are present, the amount is typically sufficient to coat the active agent(s) with at least a single molecular layer of phospholipid. In general, the phospholipid content ranges from about 5 wt % to about 99.9 wt %, such as about 20 wt % to about 80 wt %.

Generally, compatible phospholipids comprise those that have a gel to liquid crystal phase transition greater than about 40° C., such as greater than about 60° C., or greater than about 80° C. The incorporated phospholipids may be relatively long chain (e.g., C₁₆-C₂₂) saturated lipids. Exemplary phospholipids useful in the disclosed stabilized preparations include, but are not limited to, phosphoglycerides such as dipalmitoylphosphatidylcholine, distearoylphosphatidylcholine, diarachidoylphosphatidylcholine, dibehenoylphosphatidylcholine, diphosphatidyl glycerols, short-chain phosphatidylcholines, hydrogenated phosphatidylcholine, E-100-3 (available from Lipoid KG, Ludwigshafen, Germany), long-chain saturated phosphatidylethanolamines, long-chain saturated phosphatidylserines, long-chain saturated phosphatidylglycerols, long-chain saturated phosphatidylinositols, phosphatidic acid, phosphatidylinositol, and sphingomyelin.

Examples of metal ions include, but are not limited to, divalent cations, including calcium, magnesium, zinc, iron, and the like. For instance, when phospholipids are used, the pharmaceutical composition may also comprise a polyvalent cation, as disclosed in WO 01/85136 and WO 01/85137, which are incorporated herein by reference in their entireties. The polyvalent cation may be present in an amount effective to increase the melting temperature (Tₘ) of the phospholipid such that the pharmaceutical composition exhibits a Tₘ which is greater than its storage temperature (Tₛ) by at least about 20° C., such as at least about 40° C. The molar ratio of polyvalent cation to phospholipid may be at least about 0.05:1, such as about 0.05:1 to about 2.0:1 or about 0.25:1 to about 1.0:1. An example of the molar ratio of polyvalent cation:phospholipid is about 0.50:1. When the polyvalent cation is calcium, it may be in the form of calcium chloride. Although metal ion, such as calcium, is often included with phospholipid, none is required.

As noted above, the dry powder may include one or more surfactants. For instance, one or more surfactants may be in the liquid phase with one or more being associated with solid particles or particulates of the composition. By “associated with” it is meant that the pharmaceutical compositions may incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and may be used to provide the desired preparations.

Examples of nonionic detergents include, but are not limited to, sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.), which is incorporated by reference herein in its entirety.

Examples of block copolymers include, but are not limited to, diblock and triblock copolymers of polyoxyethylene and polyoxypropylene, including poloxamer 188 (Pluronic™ F-68), poloxamer 407 (Pluronic™ F-127), and poloxamer 338.

Examples of ionic surfactants include, but are not limited to, sodium sulfosuccinate, and fatty acid soaps.

Examples of amino acids include, but are not limited to, hydrophobic amino acids. Use of amino acids as pharmaceutically acceptable excipients is known in the art as disclosed in WO 95/31479, WO 96/32096, and WO 96/32149, which are incorporated herein by reference.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins.

Examples of buffers include, but are not limited to, tris or citrate.

Examples of acids include, but are not limited to, carboxylic acids.

Examples of salts include, but are not limited to, sodium chloride, salts of carboxylic acids, (e.g., sodium citrate, sodium ascorbate, magnesium gluconate, sodium gluconate, tromethamine hydrochloride, etc.), ammonium carbonate, ammonium acetate, ammonium chloride, and the like.

Examples of organic solids include, but are not limited to, camphor, and the like.

The dry powders of one or more embodiments of the present invention may also include a biocompatible polymer, such as biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Those skilled in the art will appreciate that, by selecting the appropriate polymers, the delivery efficiency of the composition and/or the stability of the dispersions may be tailored to optimize the effectiveness of the active agent(s).

Besides the above mentioned pharmaceutically acceptable excipients, it may be desirable to add other pharmaceutically acceptable excipients to the dry powder to improve particulate rigidity, production yield, emitted dose and deposition, shelf-life, and patient acceptance. Such optional pharmaceutically acceptable excipients include, but are not limited to: coloring agents, taste masking agents, buffers, hygroscopic agents, antioxidants, and chemical stabilizers. Further, various pharmaceutically acceptable excipients may be used to provide structure and form to the particulate compositions (e.g., latex particles). In this regard, it will be appreciated that the rigidifying components can be removed using a post-production technique such as selective solvent extraction.

The dry powder may also include mixtures of pharmaceutically acceptable excipients. For instance, mixtures of carbohydrates and amino acids are within the scope of the present invention.

The preparation may also include an antimicrobial agent for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant may be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases may be present as an excipient in the preparation. Nonlimiting examples of acids that may be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the active agent in the composition may vary depending on a number of factors, but may optimally be a therapeutically effective dose when the composition is stored in a unit dose container. A therapeutically effective dose may be determined experimentally by repeated administration of increasing amounts of the active agent in order to determine which amount produces a clinically desired endpoint.

The active agent may be present in the composition in an amount of about 1% to about 99% by weight, preferably from about 5%-98% by weight, more preferably from about 15-95% by weight of the active agent, with concentrations less than 30% by weight more preferred.

The amount of any individual excipient in the composition may vary depending on the activity of the excipient and particular needs of the composition. The optimal amount of any individual excipient may be determined through routine experimentation, i.e., by preparing compositions containing varying amounts of the excipient (ranging from low to high), examining the stability and other parameters, and then determining the range at which optimal performance is attained with no significant adverse effects.

The excipient may be present in the composition in an amount of about 1% to about 99% by weight, preferably from about 5%-98% by weight, more preferably from about 15-95% by weight of the excipient, with concentrations less than 30% by weight more preferred.

In one embodiment, the composition may comprise a dry powder pharmaceutical composition comprising, in percent by weight: from about 60% to about 95% insulin; and from about 5% to about 30% buffer; wherein when the composition is dissolved at a concentration of 1 mg/ml in distilled water to form a solution, the solution has a pH greater than or equal to 7.5.

In another embodiment, the composition may comprise a dry powder pharmaceutical composition comprising, in percent by weight: from about 60% to about 95% insulin; from about 5% to about 30% buffer; wherein when the composition is dissolved in an equal weight of water, has a pH greater than or equal to 7.5; and which, when exposed to an environment of 85° C. at 50% relative humidity for a period of 72 hours, exhibits less degradation, as measured by presence of a high-molecular-weight protein (HMWP) degradation product, than a dry powder insulin formulation consisting of 60 wt % human recombinant insulin, 27.06 wt % sodium citrate dehydrate, 10.01 wt % mannitol, 2.60 wt % glycine, and 0.33 wt % sodium hydroxide, tested under identical environmental conditions.

In another embodiment, the composition may comprise a powder, comprising: 85-95 wt %, on a dry basis, insulin; 5-15 wt %, on a dry basis, stabilizing excipient; 0.001-0.2 wt %, on a dry basis, alcohol; and less than 5 wt % water.

Other US patents and applications that refer to powder compositions, methods of preparing the same and methods of using the same, e.g., U.S. Pat. Nos. 6,685,967, 5,997,848, 5,826,633, 6,267,155, 6,581,650, 6,182,712, U.S. patent application Ser. Nos. 60/392,076, 10/609,132, 08/207,472, 08/383,475, 09/210,313, 09/665,2910/160,229, 10/418,966, 11/146,950, 60/100,437, 10/360,603, 60/854,601, 60/906,677, and the PCT application entitled, “Powder Dispersion Apparatus and Method of Making and Using the Apparatus, filed 25 Oct. 2007 and assigned to Nektar Therapeutics, all of which are hereby incorporated in their entirety.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C. (2000).

### EXPERIMENTAL

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All chemical reagents referred to in the appended examples are commercially available unless otherwise indicated.

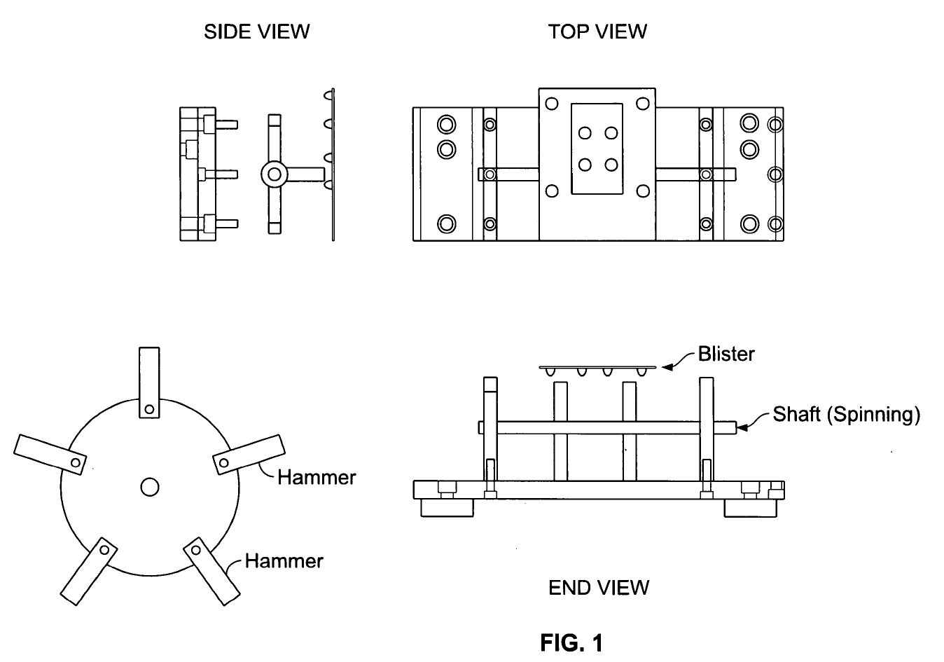

In one embodiment, the web whacker, the web containing the sealed blisters is gently tapped or whacked before it is drawn and punched into individual blisters. Collapsible arms on a circular shaft that is connected to a motor hit the underneath of the web (FIG. 1). The rotation speed of the shaft and the duration between each ‘draw’ on the packaging line determines the degree of puck break up. The duration of subjecting the web to whacking is a balance between production capacity (draw time) and efficiently breaking the puck into dispersible powder.

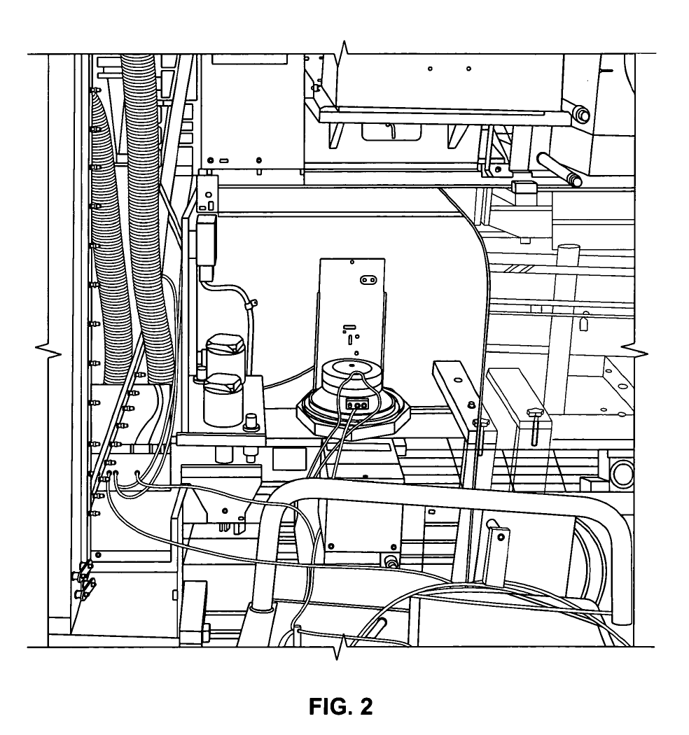

In a second embodiment, called acoustic conditioning, the web containing the sealed blisters is subjected to mechanical vibration by an acoustic speaker before it is drawn and punched into individual blisters. The speaker is located above the web (see FIG. 2). The vibration of the web can be adjusted by tuning the frequency and amplitude of the speaker which in turn is controlled by the voltage. The duration of subjecting the web to acoustic vibration is a balance between production capacity (draw time) and efficiently breaking the puck into dispersible powder.

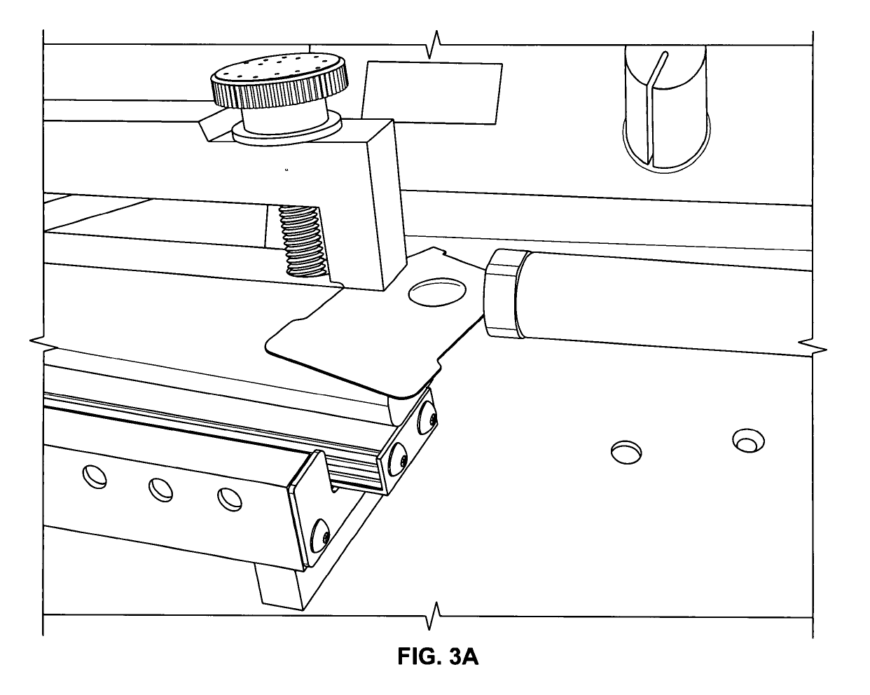

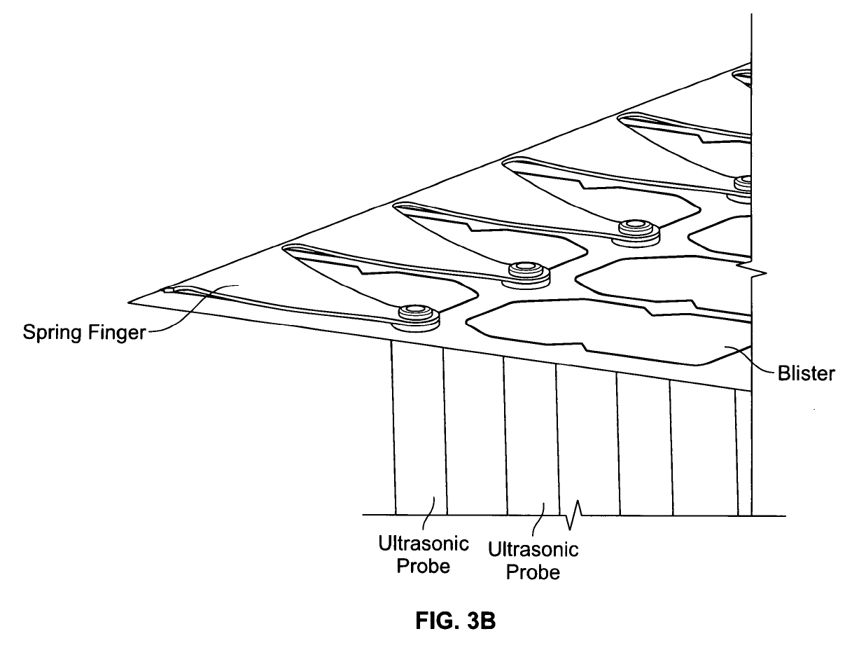

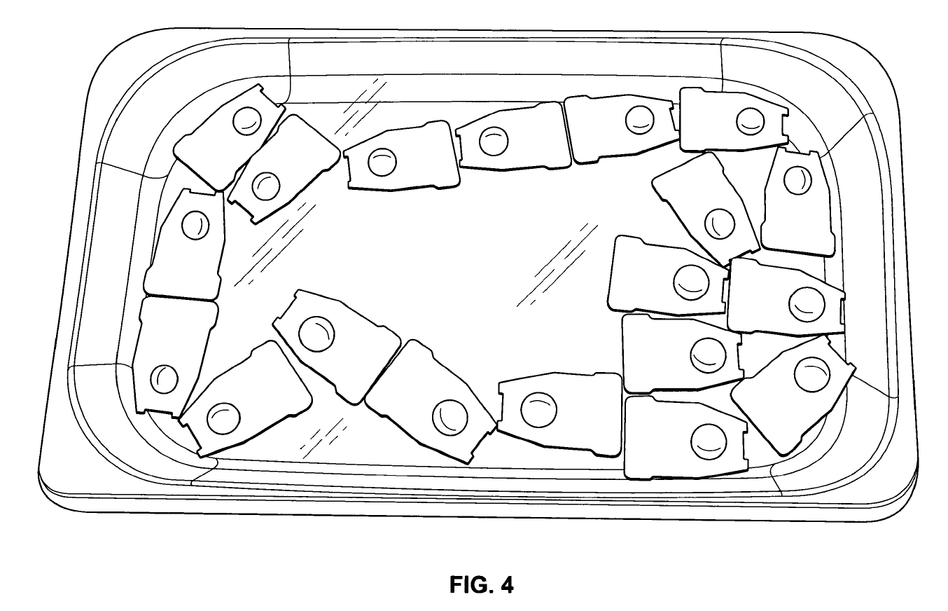

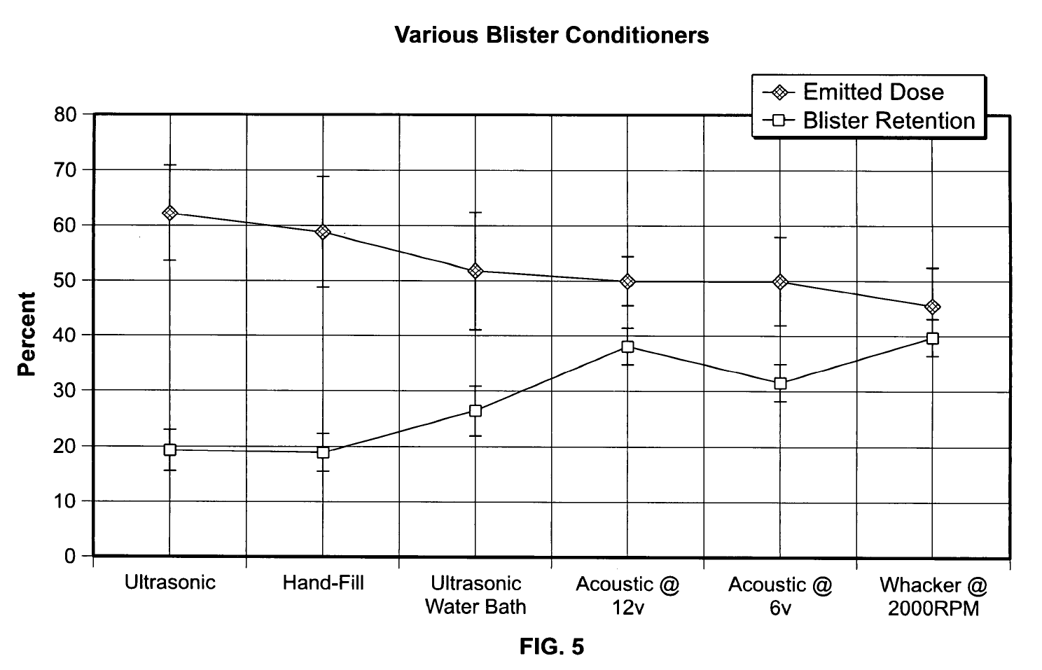

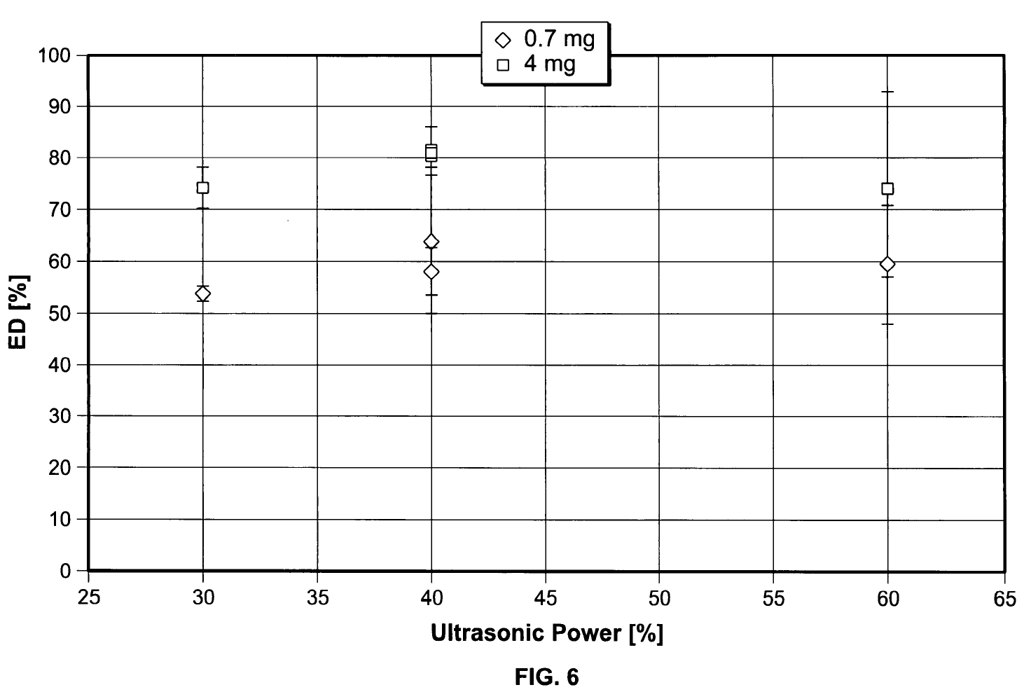

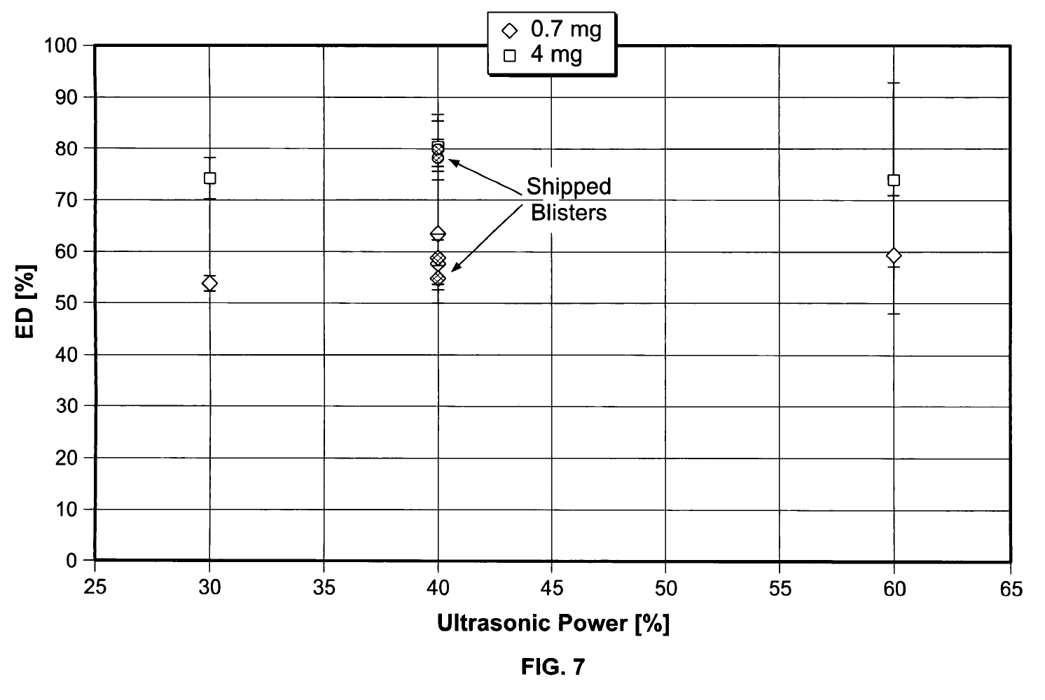

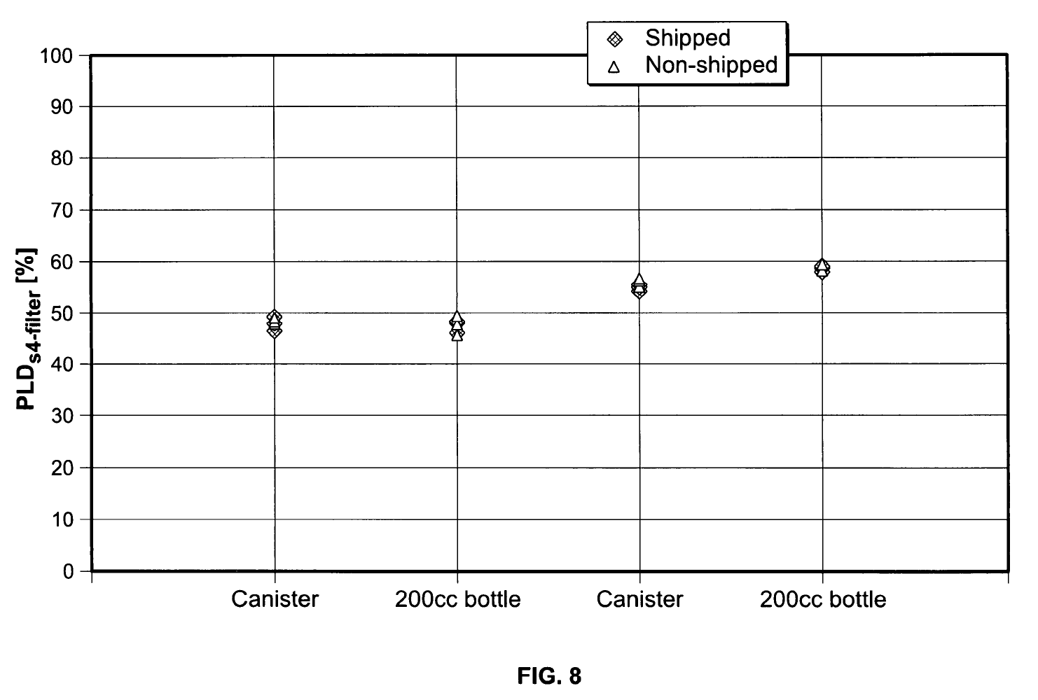

In a third embodiment, called ultrasonic conditioning, the web containing the sealed blisters is subjected to mechanical vibration by an ultrasonic probe (or an ultrasonic horn) before it is drawn and punched into individual blisters. The probe is located beneath the web (see FIG. 3A). The vibration of the web can be adjusted by tuning the amplitude of the ultrasonic probe at a fixed frequency. The efficiency of breaking the puck depends on coupling the probe with the web. The duration of subjecting the web to ultrasonic probe is a balance between production capacity (draw time) and efficiently breaking the puck into dispersible powder. The flexibility of this approach is that the probe could be located either beneath, top or on the side of the web. FIG. 3B shows a multiple ultrasonic probe-containing embodiment. FIGS. 4-8 shows the results of ultrasonic conditioning on the emitted dose of blisters under various parameters. As can be seen 40% amplitude appears to provide better conditioning, however, other power setting are also effective. It may also be seen, that once thus conditioned, shipping does not affect the emitted dose.

In a fourth embodiment, also called ultrasonic conditioning, a Branson Sonicator water bath is used, Model 2150). The water bath is filled with water to appropriate level. The dry powder blisters are placed on top of the water so that they float on top (FIG. 4). The sonicator is turned on to subject blisters to ultrasonication (40 kHz) for a settable period of time (e.g., for 1 to 5 minutes). Following sonication, blisters are wiped dry and emitted dose is compared with unsonicated blisters. The vibration of the web is determined by the frequency and amplitude of the liquid level in the bath. The duration of subjecting the web to ultrasonic probe is a balance between production capacity (draw time) and efficiently breaking the puck into dispersible powder.

1. A method of conditioning contents of at least one unit dose drug package prior to a unit dose drug package finishing step, comprising:
effecting a contact between at least one ultrasonic probe and at least one unit dose drug package to produce a vibration and at least partially deagglomerate the contents of at least one unit dose drug package.

2-3. (canceled)

4. The method of claim 1, wherein the ultrasonic probe generates a vibration frequency ranging from about 5 kHz to about 100 kHz.

5. (canceled)

6. The method of claim 1, wherein the ultrasonic probe generates a vibration amplitude of about 0.0005 inch to about 0.005 inch.

7. The method of claim 1, wherein vibration from the ultrasonic probe is applied for a variable period of time.

8-11. (canceled)

12. The method of claim 1, wherein the vibration is adjusted by tuning at least one of a frequency and amplitude of the ultrasonic probe.

13. The method of claim 1, wherein at least one unit dose drug package is urged into contact with the ultrasonic probe by a member.

14-15. (canceled)

16. The method of claim 1, wherein the ultrasonic probe is positioned beneath, above, or on a side of at least one unit dose drug package.

17. (canceled)

18. The method of claim 1, wherein the contacting comprises:
actuating a hold-down device between an engaged position in which the hold-down device directly or indirectly contacts at least one unit dose drug package, and a disengaged position in which the hold-down device does not contact a unit dose drug package.

19. The method of claim 18, wherein the contacting comprises:
vertically actuating a hold-down device between an engaged position in which the hold-down device directly or indirectly contacts at least one unit dose drug package, and a disengaged position in which the hold-down device does not contact a unit dose drug package.

20. (canceled)

21. The method of claim 1, wherein the contents comprise a dry powder medicament.

22. (canceled)

23. The method of claim 1, wherein the unit dose drug package comprises at least one blister pack.

24-29. (canceled)

30. The method of claim 1, further comprising:
filling a cavity of at least one unit dose drug package with the contents; and
sealing the filled unit dose drug package cavity to form the unit dose drug package.

31. The method of claim 30, wherein the sealing of at least one filled unit dose drug package comprises sealing a lid to the unit dose drug package cavity to form the unit dose drug package.

32-44. (canceled)

45. A method of conditioning contents of at least one unit dose drug package prior to a unit dose drug package finishing step, comprising:
contacting the at least one unit dose drug package with an ultrasonic bath to at least partially deagglomerate the contents of at least one unit dose drug package.

46-58. (canceled)

59. An apparatus for conditioning contents of at least one unit dose drug package prior to a unit dose drug package finishing step, comprising:
at least one ultrasonic probe that vibrates at least one unit dose drug package to at least partially deagglomerate the contents of the unit dose drug package.

60. The apparatus of claim 59, wherein the ultrasonic probe comprises a probe tip that vibrates of at least one unit dose drug package.

61-62. (canceled)

63. The apparatus of claim 59, wherein the unit dose drug package is a web comprising a plurality of unit dose drug packages, and wherein the ultrasonic probe comprises a plurality of ultrasonic probes structured and arranged to apply ultrasonic energy to the web comprising a plurality of unit dose drug packages.

64-70. (canceled)

71. A method of aerosolizing contents of at least one unit dose drug package, comprising:
conditioning at least one unit dose drug package according to claim 1, prior to a unit dose drug packaging step;
packaging the unit drug package; and
aerosolizing the contents of the unit dose drug package.

72. (canceled)
